(19) United States
(12) Patent Application Publication
(10) Pub. No.: US 2019/0240144 A1
(43) Pub. Date: Aug. 8, 2019

(54) EFFERVESCENT DEFERIPRONE TABLET

(71) Applicant: Khashayar KARIMIAN, Tehran (IR)

(72) Inventor: Siamak MIRTORABI, Tehran (IR)

(21) Appl. No.: 16/301,742

(22) PCT Filed: Aug. 17, 2016
(86) PCT No.: PCT/IB2016/054914

§ 371 Date: Nov. 15, 2018

(30) Foreign Application Priority Data
May 18, 2016 (IR) 13955014000300222

(51) Int. Cl. A61K 9/46; A61K 31/4412; A61K 9/00; A61K 9/20

(52) U.S. Cl. CPC A61K 9/0007; A61K 31/4412; A61K 9/2009; A61K 9/2018; A61K 9/2054; A61K 9/006

(57) ABSTRACT

An oral pharmaceutical formulation for effervescent tablet of deferiprone is reported comprising of deferiprone and a taste-masking composition consisting of an effective amount of sweetener and flavoring agent and containing appropriate amount of alkali earth metal bicarbonate or carbonate and an organic carboxylic to afford a final acid pH of about 3.

### FIELD OF THE INVENTION

This invention relates to a novel formulation of deferiprone for oral administration

### BACKGROUND

Oral administration of drugs is the most convenient and therefore preferred method of transfer of medicine into the body. Injection, on the other hand, is the least patient-compliant method. Accordingly, extensive research is carried out to formulate various drugs into orally bioavailable form. For example, cefuroxime axcetil was found to have little bioavailability in its crystalline from and it was administered in injectable from to deliver the drug into the patient's blood stream (U.S. Pat. No. 5,847,118). Later on, however, it was discovered that its amorphous form has oral bioavailability, possibly due a considerable increase in the surface area of the amorphous powder, which upon contact with the large amphiphilic surface area of the microvilli of the small intestine becomes absorbed through a surface-surface phenomenon (J Med Chem. 1998, 41, 5382-92.).

Beta thalassemia is hereditary blood disorder which is a result of imbalanced beta-chain biosynthesis of hemoglobin. This results in reduced or absent beta chain, leading to early destruction of RBCs and anemia. To remedy the situation, patients with thalassemia major become transfusion-dependent with subsequent iron overload. Excess iron results in the formation of reactive oxygen species (ROS) that cause irreversible damage to cells and organs of the patients. Accordingly, excess iron must be removed with iron chelators for the clinical management of thalassemia major (Karimian et al. Expert Opinion, Therap. Patents, 2011, 21, 819-856)

For many years, deferoxamine was the only clinically approved iron chelator available for the treatment of beta-thalassemia. Because of its high molecular weight, deferoxamine is not orally bioavailable and has to be injected subcutaneously. Furthermore, due to its low half-life (t_{1/2}=5-10 minus), the drug has to be administered over a 12 hour period using a peristaltic pump. Combined, the mode and the extended time of administration results in a very low patient compliance. Accordingly, extensive research was carried out to discover a safe and effective oral drug for the treatment of beta-thalassemia. Deferiprone, a bidentate iron chelator, was approved for clinical use as second line therapy in 1999 in Europe and in 2012 in the US. 2011. The brand product is available as 500-mg film coated tablets.

In 2011, enteric-coated tablet, which was shown to eliminate the gastrointestinal side effects of Deferiprone, was introduced by Avicenna Laboratories Inc. (Iranian Patent 71,996). The contention of elimination of gastrointestinal side effect by enteric coating the tables was based on possible effect of concentration of the drug in the stomach of the patients. The process of dissolution of the drug in the stomach may create an ever increasing concentration of the drug's active pharmaceutical ingredient in a small area of the stomach. The local concentration of the chemical may then lead to nausea, the most commonly observed gastrointestinal side effect of deferiprone. This hypothesis was tested in a single blind study of enteric-coated Deferiprone tables with 100 thalassemia major patients, who refused to take the medicine because of its gastrointestinal effect-mainly nausea. It was surprisingly discovered that 99 patients did not report any gastrointestinal side effects (Azarkeyvan A., Karimian K., Mirtorabi Mahabadi S., Narenjian A., Eslami M. Evaluation of gastric side effects of new form of deferiprone, (L1; Enteric coated), 12^{th} International Conference on Thalassemia and Other Hemoglobinopathie, the 14^{th} Thalassemia International Foundation Conference Abstract 350, 11-15 May, 2011, Anatalya, Turkey).

Exjade is the second clinically approved orally bioavailable iron chelator. This tridentate iron chelator was approved in the US and Europe in 2011 and was launched by Novartis. It is taken by patients as a suspension.

### DETAILED DESCRIPTION

Deferiprone has been shown to be very effective in the removal of excess iron from the heart. This is particularly important because myocardial siderosis, caused by excess iron in the heart, is the major cause of death in beta-thalassemia patents.

Children show low compliance to oral administration of drugs in tablet or capsule form. This is especially true when the drug dose is large, as is the case with oral iron chelators. In fact, most oral medicine for children are formulated in form of suspension or syrups (e.g. erythromycin, . . . ). Due to its efficacy in the removal of excess iron from the heart, and to make the drug available for pediatric use, Apotex Inc. formulated deferiprone in the form of syrup in 2009. Studies aimed at evaluation of deferiprone for pediatric use in children from 2 to 10 years of age is funded by the EU and is being carried out in a number of centers in Europe and elsewhere (DEEP.com). Ferriprox syrup is available in 200 ml bottles, 100 mg/ml. Dosing of the syrup in three equal portions per day using the appropriate dose of 75 mg/kg/day based the weight if the patient.

It became evident to us that formulation of deferiprone in an effervescent form would have a number of important advantages over its liquid form. First, it is well known that medicines are considerably more stable in their solid rather than liquid form. Second, medicines are much less likely to experience microbial growth in their solid form as compared to their liquid from since microbial growth require water, as in syrups or solutions (http://www.medscape.com/viewarticle/819276), and third, carrying a 200 ml bottle (https://www.medicines.org.uk/emc/medicine/21406) at all times by a patient is much more inconvenient that carrying a blister pack of 2 tablets that can be converted to palatable liquid form by simple dissolution the tablet in a glass of water. In light of the above, we now report a novel formulation of Deferiprone as effervescent tablet.

### EXAMPLES

An oral effervescent pharmaceutical formulation comprising of deferiprone, a sweetener, a fruit flavor and an alkali earth metal bicarbonate or carbonate, an organic acid, a flavoring agent, a taste masking agent, and lubricant having a final pH of 2.5 to 3.5 when dissolved in water

The following examples are not intended to limit the scope of this application. These examples are provided in order to illustrate the basic concept in this application. It is clear to men of art that obvious variations to the examples provided below would not constitute novelty.

Example 1. General Procedure for the production of Deferiprone effervescent tablet. Deferiprone, Sodium Bicarbonate, Anhydrous Citric Acid, Sucrose, Sucralose, Orange Flavor, Colloidal Silicon Dioxide are mixed for 10 to 60 minutes and preferably for 30 minutes in a double cone blender. Polyethylene glycol 6000 is added and blending is continued for 5-30 minutes, and preferably for 10 minutes. The mixture is discharged and passed through a sieve (mesh 10) then pressed to form tablets and packaged in Alu-Alu blisters. The above procedure is carried out under humidity-controlled environment.

Examples 1-9. The following ratios of the active ingredient and excipients were used in various examples

| Ingredients (mg/tablet) | F₁ | F₂ | F₃ | F₄ | F₅ | F₆ | F₇ | F₈ | F₉ |
|---|---|---|---|---|---|---|---|---|---|
| Deferiprone | 500 | 500 | 500 | 500 | 500 | 500 | 500 | 500 | 500 |
| Sodium Bicarbonate | 195 | 217 | 235 | 247 | 263 | 274 | 288 | 300 | 307 |
| Anhydrous Citric Acid | 1750 | 1700 | 1700 | 1735 | 1675 | 1670 | 1630 | 1610 | 1600 |
| Sucrose | 400 | 400 | 400 | 400 | 400 | 400 | 400 | 400 | 400 |
| Sucralose | 130 | 130 | 130 | 130 | 130 | 130 | 130 | 130 | 130 |
| Orange Flavor | 50 | 50 | 50 | 50 | 50 | 50 | 50 | 50 | 50 |
| Colloidal Silicon Dioxide | 7 | 7 | 7 | 7 | 7 | 7 | 7 | 7 | 7 |
| Polyethylene Glycol 6000 | 100 | 100 | 100 | 100 | 100 | 100 | 100 | 100 | 100 |
| Total Weight | 3132 | 3104 | 3122 | 3169 | 3125 | 3131 | 3105 | 3097 | 3094 |

Disintegration times for the above examples are provided below:

| | F₁ | F₂ | F₃ | F₄ | F₅ | F₆ | F₇ | F₈ | F₉ |
|---|---|---|---|---|---|---|---|---|---|
| Disintegration Time (min) | 12 | 12 | 10 | 10 | 8 | 8 | 7 | 7 | 4 |

F₉ formulation was selected as the best formulation because of their physicochemical characteristics.

### References

Palm, K. et al. Evaluation of dynamic polar molecular surface area as predictor of drug absorption: comparison with other computational and experimental predictors. J Med Chem. 1998, 41, 5382-92.
Azarkeyvan A., Karimian K., Mirtorabi Mahabadi S., Narenjian A., Eslami M. Evaluation of gastric side effects of new form of deferiprone, (L1; Enteric coated), 12^{th} International Conference on Thalassemia and Other Hemoglobinopathie, the 14^{th} Thalassemia International Foundation Conference Abstract 350, 11-15 May 2011, Anatalya, Turkey.
U.S. Pat. No. 5,847,118
Karimian, K., et al. Advances in iron chelation: an update Expert Opin. Ther. Patents, 2011, 21, 819, 856
Tam, et al. Curr. Adv. Med. Chem. 2003, 10, 983-995.
Rund, D., et al., New Eng. J. Med., 2005, 353, 1135-1146
European Commission Decision of Nov. 19, 2007 (labeled “Not for Publication”) approving Apotex's Ferriprox 100mg/ml oral solution, available at http://ec.europa.eu/health/documents/community-register/html/h108.htm (Jul. 17, 2013).
Hershko, C. et al. Ann. N.Y. Acad. Sci., 2005, 1054, 124-135.

We claim is:

1. An effervescent, rapidly disintegrating oral dosage form of Deferiprone, which comprises of:
(a) an active ingredient (Deferiprone),
(b) an effervescent base, wherein said effervescent base and consists essentially of
(c) a pharmaceutically acceptable auxiliary ingredient.
(i) at least one polysaccharide sweetener,
(ii) an edible organic add,
(iii) an alkali metal salt, carbonate or bicarbonate of the said edible organic acid citric

2. The dosage form of claim 1, wherein the dosage form comprises a tablet, granules, or sachets, said dosage form being adapted to be dissolved in water before being taken.

3. The dosage form of claim 1, wherein the dosage form comprises a buccal or sublingual tablet adapted to be administered directly into the oral cavity.

4. The dosage form of claims 1, wherein said alkali-sensitive active ingredient is Deferiprone or a pharmaceutically acceptable salt thereof.

5. The dosage form of claim 4, wherein said active ingredient is Deferiprone or its pharmaceutically effective salt.

6. The dosage form of claim 1 wherein said effervescent base comprises a minimum of about 15% wt. sodium citrate.

7. The dosage form of claim 1, wherein said effervescent base comprises a maximum of about 90% wt. of the dosage form.

8. The dosage form of claim 1, said active ingredient comprises from about 30% wt. to about 70% wt. of said effervescent base.

9. The dosage form of claim 1 in which the flavoring agent selected from the group consisting of natural flavors, natural fruit flavors, artificial fruit flavors, peppermint, peppermint oil and mixtures thereof.

10. The dosage form of claim 1 in which the flavoring agent comprises of more than one agent.

11. The dosage form of claim 1, wherein the dosage forms is an effervescent minitablet comprising from about 50 mg to about 1000 mg Deferiprone, and about 1200 mg of said effervescent base.

12. The dosage form of claim 1, wherein the dosage form is a buccal preparation comprising from about 50 to about 1000 mg Deferiprone and from about 100 to about 5000 mg of said effervescent base.

13. The dosage form of claim 13, wherein said buccal preparation comprises from about 50 to about 1000 mg Deferiprone.

14. The dosage form of claim 1, wherein said auxiliary ingredient comprises at least one of (i) a colorant, and (ii) at least one sweetener.

15. The dosage form of claim 15, wherein said sweetener is at least one of a sucrose, xylitol, D-glucose, sorbitol, mannitol, lactose, aspartame, sodium saccharine, acesulfam, and sodium cyclamate.

16. A process for preparing the dosage form of claim 1, which comprises separately granulating said polysaccharide and said organic edible acid, and adding said alkali-sensitive active ingredient to the acidic granules, and if necessary drying the granules, and mixing the dried granules.

17. The process of claim 17, wherein said auxiliary ingredient is at least one of lactose, sucrose, sorbitol, mannitol, starch, pectin, or cellulose.

18. A process for preparing the dosage form of claim 1, which comprises mixing all of the active ingredients and of said effervescent base, heating and drying the mixture.
